(19) United States
(12) Patent Application Publication
(10) Pub. No.: US 2025/0009706 A1
(43) Pub. Date: Jan. 9, 2025

(54) COMPOSITIONS AND METHODS FOR TREATING OPIOID DEPENDENCE AND WITHDRAWAL

(71) Applicant: CASE WESTERN RESERVE UNIVERSITY, Cleveland, OH (US)

(72) Inventor: Stephen J. Lewis, Cleveland, OH (US)

(21) Appl. No.: 18/580,474

(22) PCT Filed: Jul. 18, 2022
(86) PCT No.: PCT/US2022/037465

§ 371 Date: Jan. 18, 2024

Related U.S. Application Data
(60) Provisional application No. 63/223,011, filed on Jul. 18, 2021.

(51) Int. Cl. A61K 31/223; A61K 31/225; A61K 31/485; A61P 25/36

(52) U.S. Cl. CPC A61K 31/223; A61K 31/225; A61K 31/485; A61P 25/36

(57) ABSTRACT

A method of treating opioid withdrawal and/or dependence in a subject in need thereof, includes administering to the subject a therapeutically effective amount of a composition comprising a D-cysteine ester, a cystine ester, or an adduct, a pharmaceutically acceptable salt, a tautomer, or a solvate thereof.

### RELATED APPLICATION

This application claims priority from U.S. Provisional Application No. 63/223,011 filed Jul. 18, 2021, the subject matter of which are incorporated herein by reference in their entirety.

### BACKGROUND

Substance addiction is a serious public health problem throughout the world. Heroin and other opioids, including prescription painkillers, are widely abused and account for a large percentage of illicit drug use. Opioid use is also linked to approximately 50% of violent crimes in the United States and costs the U.S. economy billions of dollars per year.

Acute withdrawal from drug dependence is characterized by dramatic and traumatic symptoms, including sweating, racing heart, palpitations, muscle tension, tightness in the chest, difficulty breathing, tremor, nausea, vomiting, diarrhea, grand mal seizures, heart attacks, strokes, hallucinations and delirium tremens (DTs). Once acute withdrawal symptoms have subsided, post-acute withdrawal syndrome can last for months or years. Post-acute withdrawal symptoms include fatigue, depression, lack of motivation, and increased pain sensitivity.

Numerous treatments have been developed in attempts to ameliorate acute and post-acute withdrawal symptoms. However, in most cases, treatment of withdrawal requires use of other addictive substances (e.g., morphine or methadone). Treatment also requires that the addict attend a clinic daily for an extended amount of time. Due to the severity and duration of withdrawal symptoms, opioid-addicted patients have a high rate of relapse. There is a significant need for effective, non-addictive treatment for acute and post-acute opioid withdrawal symptoms.

### SUMMARY

Embodiments described herein relate to compositions and methods of attenuating and/or treating opioid withdrawal and/or dependence in a subject in need thereof, and particularly relates to the use of thiol-based compounds in compositions and methods of attenuating and/or treating opioid withdrawal and/or dependence.

In some embodiments, the thiol-based compound can include a D-cysteine ester, a cystine ester, or an adduct, a pharmaceutically acceptable salt, a tautomer, or a solvate thereof and the opioid withdrawal and/or dependence can be attenuated and/or treated by administering to the subject a therapeutically effective amount of the cysteine ester, cystine ester, or adduct, pharmaceutically acceptable salt, tautomer, or solvate thereof.

In some embodiments, the subject has an opioid dependence or addiction.

In other embodiments, the subject has neonatal opioid withdrawal syndrome.

In some embodiments, the D-cysteine alkyl ester includes a compound having the structure of the formula:

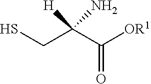

or a pharmaceutically acceptable salt, tautomer, or solvate thereof; where R¹ is an unsubstituted or substituted lower alkyl (e.g., C₁-C₆ alkyl).

In some embodiments, the D-cysteine alkyl ester comprises a D-cysteine ethyl ester, adduct thereof, or pharmaceutically acceptable salt, tautomer, or solvate thereof.

In some embodiments, the adduct of the D-cysteine alkyl ester comprises at least one of an albumin adduct, a glucose adduct, an L-cysteine adduct, an L-glutathione adduct, and an S-nitroso adduct.

In other embodiments, the cystine ester has the formula:

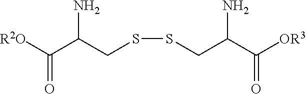

or an adduct, a pharmaceutically acceptable salt, a tautomer, or a solvate thereof; where R² and R³ are the same or different and are selected from the group consisting of H, unsubstituted or substituted C₁-C₂₄ alkyl, C₂-C₂₄ alkenyl, C₂-C₂₄ alkynyl, C₃-C₂₀ aryl, heterocycloalkenyl containing from 5-6 ring atoms, heteroaryl, and heterocyclyl containing from 5-14 ring atoms, wherein at least one of R² and R³ is not a H.

In some embodiments, R² and R³ are independently H or an unsubstituted or substituted C₁-C₂₄ alkyl, wherein at least one of R² and R³ is not a H.

In some embodiments, R² and R³ are independently selected from the group consisting of H, methyl, ethyl, propyl, and butyl, wherein at least one of R² and R³ is not a H.

In some embodiments, the cystine ester is a cystine dialkyl ester. For example, the cystine dialkyl ester can be a D-cystine dialkyl ester or an adduct, a pharmaceutically acceptable salt, a tautomer, or a solvate thereof.

In some embodiments, the cystine dialkyl ester is selected from the group consisting of cystine dimethyl ester, cystine diethyl ester, combinations thereof, or an adduct, a pharmaceutically acceptable salt, a tautomer, or a solvate thereof.

In some embodiments, the cystine dialkyl ester is D-cystine dimethyl ester or an adduct, a pharmaceutically acceptable salt, a tautomer, or a solvate thereof.

In some embodiments, the opioid can include at least one of alfentanil, buprenorphine, butorphanol, carfentanil, codeine, diamorphine, dextromoramide, dezocine, dihydrocodeine, fentanyl, hydrocodone, hydromorphone, levorphanol, meperidine, meptazinol, methadone, morphine, nalbuphine, nalorphine, opium, oxycodone, oxymorphone, pentazocine, propoxyphene, remifentanil, sufentanil, tapentadol, and tramadol, and pharmaceutically acceptable salts thereof. For example, the opioid can be carfentanil, fentanyl, remifentanil, or sufentanil. In another example, the opioid can include morphine and/or fentanyl.

In other embodiments, the method further includes administering an opioid antagonist to the subject.

In some embodiments, the opioid antagonist is naloxone, an oxymorphol analog of naloxone, a naloxone salt, or a naloxone dihydrate.

In some embodiments, the opioid antagonist is naloxone.

In some embodiments, the oxymorphol analog of naloxone is naltrexone.

Other embodiments described herein relate to a method of treating neonatal opioid withdrawal syndrome in a subject in need thereof. The method includes administering to the subject a therapeutically effective amount of a composition comprising a D-cysteine ester, a cystine ester, or an adduct, a pharmaceutically acceptable salt, a tautomer, or a solvate thereof.

In some embodiments, the D-cysteine alkyl ester includes a compound having the structure of the formula:

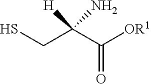

or a pharmaceutically acceptable salt, tautomer, or solvate thereof; where R¹ is an unsubstituted or substituted lower alkyl(C₁-C₆ alkyl).

In some embodiments, the D-cysteine alkyl ester comprises a D-cysteine ethyl ester, adduct thereof, or pharmaceutically acceptable salt, tautomer, or solvate thereof.

In some embodiments, the adduct of the D-cysteine alkyl ester comprises at least one of an albumin adduct, a glucose adduct, an L-cysteine adduct, an L-glutathione adduct, and an S-nitroso adduct.

In other embodiments, the cystine ester has the formula:

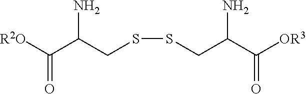

or an adduct, a pharmaceutically acceptable salt, a tautomer, or a solvate thereof; where R² and R³ are the same or different and are selected from the group consisting of H, unsubstituted or substituted C₁-C₂₄ alkyl, C₂-C₂₄ alkenyl, C₂-C₂₄ alkynyl, C₃-C₂₀ aryl, heterocycloalkenyl containing from 5-6 ring atoms, heteroaryl, and heterocyclyl containing from 5-14 ring atoms, wherein at least one of R² and R³ is not a H.

In some embodiments, R² and R³ are independently H or an unsubstituted or substituted C₁-C₂₄ alkyl, wherein at least one of R² and R³ is not a H.

In some embodiments, R² and R³ are independently selected from the group consisting of H, methyl, ethyl, propyl, and butyl, wherein at least one of R² and R³ is not a H.

In some embodiments, the cystine ester is a cystine dialkyl ester. For example, the cystine dialkyl ester can be a D-cystine dialkyl ester or an adduct, a pharmaceutically acceptable salt, a tautomer, or a solvate thereof.

In some embodiments, the cystine dialkyl ester is selected from the group consisting of cystine dimethyl ester, cystine diethyl ester, combinations thereof, or an adduct, a pharmaceutically acceptable salt, a tautomer, or a solvate thereof.

In some embodiments, the cystine dialkyl ester is D-cystine dimethyl ester or an adduct, a pharmaceutically acceptable salt, a tautomer, or a solvate thereof.

### BRIEF DESCRIPTION OF THE DRAWINGS

FIG. 1 illustrates a graph showing D-CYSee diminishes withdrawal responses in morphine dependent rats.

FIG. 2 illustrates a schematic timeline of an assay of neonatal morphine exposure of female mice for determining neonatal opioid withdrawal syndrome (NOWS).

FIG. 3 illustrates a graphs and a plot showing developmental milestones are impaired following early opioid exposure.

FIG. 4 illustrates a graph showing D-CYSee modulates NOWS.

### DETAILED DESCRIPTION

For convenience, certain terms employed in the specification, examples, and appended claims are collected here. Unless defined otherwise, all technical and scientific terms used herein have the same meaning as commonly understood by one of ordinary skill in the art to which this application belongs.

The articles “a” and “an” are used herein to refer to one or to more than one (i.e., to at least one) of the grammatical object of the article. By way of example, “an element” means one element or more than one element.

The terms “comprise,” “comprising,” “include,” “including,” “have,” and “having” are used in the inclusive, open sense, meaning that additional elements may be included. The terms “such as”, “e.g.,” as used herein are non-limiting and are for illustrative purposes only. “Including” and “including but not limited to” are used interchangeably.

The term “or” as used herein should be understood to mean “and/or”, unless the context clearly indicates otherwise.

The term “about” or “approximately” as used herein refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

It will be noted that the structure of some of the compounds of the application include asymmetric (chiral) carbon or sulfur atoms. It is to be understood accordingly that the isomers arising from such asymmetry are included herein, unless indicated otherwise. Such isomers can be obtained in substantially pure form by classical separation techniques and by stereochemically controlled synthesis. The compounds of this application may exist in stereoisomeric form, therefore, can be produced as individual stereoisomers or as mixtures.

The term “isomerism” refers to compounds that have identical molecular formulae but that differ in the nature or the sequence of bonding of their atoms or in the arrangement of their atoms in space. Isomers that differ in the arrangement of their atoms in space are termed “stereoisomers”. Stereoisomers that are not mirror images of one another are termed “diastereoisomers”, and stereoisomers that are non-superimposable mirror images are termed “enantiomers”, or sometimes optical isomers. A carbon atom bonded to four nonidentical substituents is termed a “chiral center” whereas a sulfur bound to three or four different substituents, e.g., sulfoxides or sulfinimides, is likewise termed a “chiral center”.

The term “chiral isomer” refers to a compound with at least one chiral center. It has two enantiomeric forms of opposite chirality and may exist either as an individual enantiomer or as a mixture of enantiomers. A mixture containing equal amounts of individual enantiomeric forms of opposite chirality is termed a “racemic mixture”. A compound that has more than one chiral center has 2n-1 enantiomeric pairs, where n is the number of chiral centers. Compounds with more than one chiral center may exist as either an individual diastereomer or as a mixture of diastereomers, termed a “diastereomeric mixture”. When one chiral center is present, a stereoisomer may be characterized by the absolute configuration (R or S) of that chiral center. Alternatively, when one or more chiral centers are present, a stereoisomer may be characterized as (+) or (−). Absolute configuration refers to the arrangement in space of the substituents attached to the chiral center. The substituents attached to the chiral center under consideration are ranked in accordance with the Sequence Rule of Cahn, Ingold and Prelog. (Cahn et al, Angew. Chem. Inter. Edit. 1966, 5, 385; errata 511; Cahn et al., Angew. Chem. 1966, 78, 413; Cahn and Ingold, J Chem. Soc. 1951 (London), 612; Cahn et al., Experientia 1956, 12, 81; Cahn, J., Chem. Educ. 1964, 41, 116).

The term “geometric isomers” refers to diastereomers that owe their existence to hindered rotation about double bonds. These configurations are differentiated in their names by the prefixes cis and trans, or Z and E, which indicate that the groups are on the same or opposite side of the double bond in the molecule according to the Cahn-Ingold-Prelog rules. Further, the structures and other compounds discussed in this application include all atropic isomers thereof.

The term “atropic isomers” refers to a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

The terms “addiction” and “dependence” are used interchangeably to refer to the patient's inability to stop using the opioid or opioid-like drug, even when it would be in his/her best interest to stop. The Diagnostic and Statistical Manual of Mental Disorders Text Revision (DSMIV-TR) criteria for dependency include: Dependence or significant impairment or distress, as manifested by 3 or more of the following during a 12 month period: 1. Tolerance or markedly increased amounts of the substance to achieve intoxication or desired effect or markedly diminished effect with continued use of the same amount of substance. 2. Withdrawal symptoms or the use of certain substances to avoid withdrawal symptoms. 3. Use of a substance in larger amounts or over a longer period than was intended. 4. Persistent desire or unsuccessful efforts to cut down or control substance use. 5. Involvement in chronic behavior to obtain the substance, use the substance, or recover from its effects. 6. Reduction or abandonment of social, occupational or recreational activities because of substance use. 7. Use of substances even though there is a persistent or recurrent physical or psychological problem that is likely to have been caused or exacerbated by the substance.

“Drug withdrawal” refers to a group of symptoms that occur upon the abrupt discontinuation or sudden decrease in intake of medications or recreational drugs. Consequently, “opioid withdrawal” refers to the group of symptoms that occur upon the dramatic reduction, abrupt discontinuation or decrease in intake of opioids or opiates. Withdrawal symptoms may also start between doses. Withdrawal symptoms from opioids include but are not limited to anxiety, depression, sweating, vomiting, and diarrhea, muscle cramping, agitation, insomnia, yawning dilated pupils, goose bumps, abdominal cramping, runny nose and increased tearing, for example.

The term “opioid” refers to naturally-occurring opiates and synthetic or semi-synthetic opioids that have psychoactive effects. Non-limiting examples include acetyl-alpha-methylphentanyl, acetylmethadol, alfentanil, allylprodine, alphacetylmethadol, alphamethadol, alpha-methylfentanyl, alpha-methylthiofentanyl, alphaprodine, anileridine, benzylmorphine, benzethidine, betacetylmethadol, beta-hydroxyfentanyl, beta-hydroxy-3-methylfentanyl, betameprodine, betacetylmethadol, beta-hydroxyfentanyl, beta-hydroxy-3-methylfentanyl, betameprodine, betamethadol, betaprodine, bezitramide, buprenorphine, butorphanol, carfentanil, clonitazene, codeine, desomorphine, dextromoramide, dextropropoxyphene, dezocine, diampromide, diamorphone, diethylthiambutene, dihydrocodeine, dihydroetorphine, dihydromorphine, dimenoxadol, dimepheptanol, dimethyl-thiambutene, dioxaphetyl butyrate, diphenoxylate, difenoxin, dipipanone, eptazocine, ethoheptazine, ethylmethylthiambutene, ethylmorphine, etonitazene, etorphine, etoxeridine, fentanyl, furethidine, heroin, hydrocodone, hydromorphone, hydroxypethidine, isomethadone, ketobemidone, levo-alphacetylmethadol, levomethorphan, levorphanol, levophenacylmorphan, levomoranude, lofentanil, loperamide, laudanum, meperidine, meptazinol, metazocine, methadone, 3-methylfentanyl, 3-methylthiofentanyl, metopon, morphine, morpheridine, MPPP (1-methyl-4-phenyl-4-propionoxypiperidine), myrophine, narceine, nicomorphine, noracymethadol, norlevorphanol, normethadone, nalorphine, nalbuphene, normorphine, norpipanone, opium, oxycodone, oxymorphone, papaveretum, para-fluorofentanyl, paregoric, PEPAP (1-(-2-phenethyl)-4-phenyl-4-acetoxypiperidine), pentazocine, phenadoxone, phenampromide, phenomorphan, phenazocine, phenoperidine, piminodine, piritramide, propheptazine, promedol, properidine, propiram, propoxyphene, racemoramide, racemethorphan, racemorphan, remifentanil, sufentanil, tapentadol, thebaine, thiofentanyl, tilidine, tramadol, trimeperidine, mixtures of any of the foregoing, salts of any of the foregoing, derivatives of any of the foregoing, and the like. The term opioids also encompasses opioid intermediates, including 4-cyano-2-dimethylamino-4,4-diphenyl butane, 2-methyl-3-morpholino-1,1-diphenylpropane-carboxylic acid, 4-cyano-1-methyl-4-phenylpiperidine, ethyl-4-phenylpiperidine-4-carboxylate, and 1-methyl-4-phenylpiperidine-4-carboxylic acid.

Neonatal abstinence syndrome (NAS) is a complex of signs and symptoms in the postnatal period associated with the sudden withdrawal of maternally transferred opioids. The main manifestations include increased muscle tone, autonomic instability, irritability, poor sucking reflex, and impaired weight increase.

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrastemal injection and infusion.

The terms “treatment,” “treating,” and “treat” refer to acting upon a disease, disorder, or condition with an agent to reduce or ameliorate harmful or any other undesired effects of the disease, disorder, or condition and/or its symptoms. “Treatment,” as used herein, covers the treatment of a human patient, and includes: (a) reducing the risk of occurrence of the condition in a patient determined to be predisposed to the condition but not yet diagnosed as having the condition, (b) impeding the development of the condition, and/or (c) relieving the condition, i.e., causing regression of the condition and/or relieving one or more symptoms of the condition. “Treating” or “treatment of” a condition or patient refers to taking steps to obtain beneficial or desired results, including clinical results such as the reduction of symptoms. For purposes of this invention, beneficial or desired clinical results include, but are not limited to: treating opioid or opioid-like drug addiction; treating, preventing, and/or attenuating acute withdrawal symptoms; treating, preventing, and/or attenuating long-term (post-acute) withdrawal symptoms; and preventing relapse of opioid or opioid-like drug use.

The term “preventing” is art-recognized and includes stopping a disease, disorder or condition from occurring in a subject, which may be predisposed to the disease, disorder and/or condition but has not yet been diagnosed as having it. Preventing a condition related to a disease includes stopping the condition from occurring after the disease has been diagnosed but before the condition has been diagnosed.

The term “pharmaceutical composition” refers to a formulation containing the disclosed compounds in a form suitable for administration to a subject. In some embodiments, the pharmaceutical composition is in bulk or in unit dosage form. The unit dosage form is any of a variety of forms, including, for example, a capsule, an IV bag, a tablet, a single pump on an aerosol inhaler, or a vial. The quantity of active ingredient (e.g., a formulation of the disclosed compound or salts thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, intranasal, inhalational, and the like. Dosage forms for the topical or transdermal administration of a compound described herein includes powders, sprays, ointments, pastes, creams, lotions, gels, solutions, patches, nebulized compounds, and inhalants. In some embodiments, the compound or active ingredient is mixed under sterile conditions with a pharmaceutically acceptable carrier, and with any preservatives, buffers, or propellants that are required.

The term “flash dose” refers to compound formulations that are rapidly dispersing dosage forms.

The term “immediate release” is defined as a release of compound from a dosage form in a relatively brief period of time, generally up to about 60 minutes. The term “modified release” is defined to include delayed release, extended release, and pulsed release. The term “pulsed release” is defined as a series of releases of drug from a dosage form. The term “sustained release” or “extended release” is defined as continuous release of a compound from a dosage form over a prolonged period.

The phrase “pharmaceutically acceptable” is art-recognized. In certain embodiments, the term includes compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” is art-recognized, and includes, for example, pharmaceutically acceptable materials, compositions or vehicles, such as a liquid or solid filler, diluent, excipient, solvent or encapsulating material, involved in carrying or transporting any subject composition from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic. Some examples of materials, which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The compounds of the application are capable of further forming salts. All of these forms are also contemplated herein.

“Pharmaceutically acceptable salt” of a compound means a salt that is pharmaceutically acceptable and that possesses the desired pharmacological activity of the parent compound. For example, the salt can be an acid addition salt. One embodiment of an acid addition salt is a hydrochloride salt. The pharmaceutically acceptable salts can be synthesized from a parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile being preferred. Lists of salts are found in Remington's Pharmaceutical Sciences, 18th ed. (Mack Publishing Company, 1990).

The compounds described herein can also be prepared as esters, for example pharmaceutically acceptable esters. For example, a carboxylic acid function group in a compound can be converted to its corresponding ester, e.g., a methyl, ethyl, or other ester. Also, an alcohol group in a compound can be converted to its corresponding ester, e.g., an acetate, propionate, or other ester.

The compounds described herein can also be prepared as prodrugs, for example pharmaceutically acceptable prodrugs. The terms “pro-drug” and “prodrug” are used interchangeably herein and refer to any compound, which releases an active parent drug in vivo. Since prodrugs are known to enhance numerous desirable qualities of pharmaceuticals (e.g., solubility, bioavailability, manufacturing, etc.) the compounds can be delivered in prodrug form. Thus, the compounds described herein are intended to cover prodrugs of the presently claimed compounds, methods of delivering the same and compositions containing the same. “Prodrugs” are intended to include any covalently bonded carriers that release an active parent drug in vivo when such prodrug is administered to a subject. Prodrugs are prepared by modifying functional groups present in the compound in such a way that the modifications are cleaved, either in routine manipulation or in vivo, to the parent compound. Prodrugs include compounds wherein a hydroxy, amino, sulfhydryl, carboxy, or carbonyl group is bonded to any group that may be cleaved in vivo to form a free hydroxyl, free amino, free sulfhydryl, free carboxy or free carbonyl group, respectively. Prodrugs can also include a precursor (forerunner) of a compound described herein that undergoes chemical conversion by metabolic processes before becoming an active or more active pharmacological agent or active compound described herein.

Examples of prodrugs include, but are not limited to, esters (e.g., acetate, dialkylaminoacetates, formates, phosphates, sulfates, and benzoate derivatives) and carbamates (e.g., N,N-dimethylaminocarbonyl) of hydroxy functional groups, ester groups (e.g., ethyl esters, morpholinoethanol esters) of carboxyl functional groups, N-acyl derivatives (e.g., N-acetyl)N-Mannich bases, Schiff bases and enaminones of amino functional groups, oximes, acetals, ketals and enol esters of ketone and aldehyde functional groups in compounds, and the like, as well as sulfides that are oxidized to form sulfoxides or sulfones.

The term “protecting group” refers to a grouping of atoms that when attached to a reactive group in a molecule masks, reduces or prevents that reactivity. Examples of protecting groups can be found in Green and Wuts, Protective Groups in Organic Chemistry, (Wiley, 2.sup.nd ed. 1991); Harrison and Harrison et al., Compendium of Synthetic Organic Methods, Vols. 1-8 (John Wiley and Sons, 1971-1996); and Kocienski, Protecting Groups, (Verlag, 3^{rd} ed. 2003).

The term “amine protecting group” is intended to mean a functional group that converts an amine, amide, or other nitrogen-containing moiety into a different chemical group that is substantially inert to the conditions of a particular chemical reaction. Amine protecting groups are preferably removed easily and selectively in good yield under conditions that do not affect other functional groups of the molecule. Examples of amine protecting groups include, but are not limited to, formyl, acetyl, benzyl, t-butyldimethylsilyl, t-butyldiphenylsilyl, t-butyloxycarbonyl (Boc), p-methoxybenzyl, methoxymethyl, tosyl, trifluoroacetyl, trimethylsilyl (TMS), fluorenyl-methyloxycarbonyl, 2-trimethylsilyl-ethyoxycarbonyl, 1-methyl-1-(4-biphenylyl) ethoxycarbonyl, allyloxycarbonyl, benzyloxycarbonyl (CBZ), 2-trimethylsilyl-ethanesulfonyl (SES), trityl and substituted trityl groups, 9-fluorenylmethyloxycarbonyl (FMOC), nitro-veratryloxycarbonyl (NVOC), and the like. Those of skill in the art can identify other suitable amine protecting groups.

Representative hydroxy protecting groups include those where the hydroxy group is either acylated or alkylated such as benzyl, and trityl ethers as well as alkyl ethers, tetrahydropyranyl ethers, trialkylsilyl ethers and allyl ethers.

Additionally, the salts of the compounds described herein, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

The term “solvates” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate.

The compounds, salts and prodrugs described herein can exist in several tautomeric forms, including the enol and imine form, and the keto and enamine form and geometric isomers and mixtures thereof. Tautomers exist as mixtures of a tautomeric set in solution. In solid form, usually one tautomer predominates. Even though one tautomer may be described, the present application includes all tautomers of the present compounds. A tautomer is one of two or more structural isomers that exist in equilibrium and are readily converted from one isomeric form to another. This reaction results in the formal migration of a hydrogen atom accompanied by a switch of adjacent conjugated double bonds. In solutions where tautomerization is possible, a chemical equilibrium of the tautomers will be reached.

The exact ratio of the tautomers depends on several factors, including temperature, solvent, and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs.

Tautomerizations can be catalyzed by: Base: 1. deprotonation; 2. formation of a delocalized anion (e.g., an enolate); 3. protonation at a different position of the anion; Acid: 1. protonation; 2. formation of a delocalized cation; 3. deprotonation at a different position adjacent to the cation.

A “patient,” “subject,” or “host” to be treated by the compounds or methods described herein may mean either a human or non-human animal, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder.

The terms “prophylactic” or “therapeutic” treatment is art-recognized and includes administration to the host of one or more of the subject compounds. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The terms “therapeutic agent”, “drug”, “medicament”, “active ingredient”, and “bioactive substance” are art-recognized and include molecules and other agents that are biologically, physiologically, or pharmacologically active substances that act locally or systemically in a patient or subject to treat a disease or condition. The terms include without limitation pharmaceutically acceptable salts thereof and prodrugs. Such agents may be acidic, basic, or salts; they may be neutral molecules, polar molecules, or molecular complexes capable of hydrogen bonding; they may be prodrugs in the form of ethers, esters, amides and the like that are biologically activated when administered into a patient or subject.

The phrase “therapeutically effective amount” or “pharmaceutically effective amount” is an art-recognized term. In certain embodiments, the term refers to an amount of a therapeutic agent that produces some desired effect at a reasonable benefit/risk ratio applicable to any medical treatment. In certain embodiments, the term refers to that amount necessary or sufficient to eliminate, reduce or maintain a target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation. In certain embodiments, a therapeutically effective amount of a therapeutic agent for in vivo use will likely depend on a number of factors, including: the rate of release of an agent from a polymer matrix, which will depend in part on the chemical and physical characteristics of the polymer; the identity of the agent; the mode and method of administration; and any other materials incorporated in the polymer matrix in addition to the agent.

With respect to any chemical compounds, the present application is intended to include all isotopes of atoms occurring in the present compounds. Isotopes include those atoms having the same atomic number but different mass numbers. By way of general example and without limitation, isotopes of hydrogen include tritium and deuterium, and isotopes of carbon include C-13 and C-14.

When a bond to a substituent is shown to cross a bond connecting two atoms in a ring, then such substituent can be bonded to any atom in the ring. When a substituent is listed without indicating the atom via which such substituent is bonded to the rest of the compound of a given formula, then such substituent can be bonded via any atom in such substituent. Combinations of substituents and/or variables are permissible, but only if such combinations result in stable compounds.

When an atom or a chemical moiety is followed by a subscripted numeric range (e.g., C₁₋₆), it is meant to encompass each number within the range as well as all intermediate ranges. For example, “C₁₋₆ alkyl” is meant to include alkyl groups with 1, 2, 3, 4, 5, 6, 1-6, 1-5, 1-4, 1-3, 1-2, 2-6, 2-5, 2-4, 2-3, 3-6, 3-5, 3-4, 4-6, 4-5, and 5-6 carbons.

The term “alkyl” is intended to include both branched (e.g., isopropyl, tert-butyl, isobutyl), straight-chain e.g., methyl, ethyl, propyl, butyl, pentyl, hexyl, heptyl, octyl, nonyl, decyl), and cycloalkyl (e.g., alicyclic) groups (e.g., cyclopropyl, cyclopentyl, cyclohexyl, cycloheptyl, cyclooctyl), alkyl substituted cycloalkyl groups, and cycloalkyl substituted alkyl groups. Such aliphatic hydrocarbon groups have a specified number of carbon atoms. For example, C₁₋₆ alkyl is intended to include C₁, C₂, C₃, C₄, C₅, and C₆ alkyl groups. As used herein, “lower alkyl” refers to alkyl groups having from 1 to 6 carbon atoms in the backbone of the carbon chain. “Alkyl” further includes alkyl groups that have oxygen, nitrogen, sulfur or phosphorous atoms replacing one or more hydrocarbon backbone carbon atoms. In certain embodiments, a straight chain or branched chain alkyl has six or fewer carbon atoms in its backbone (e.g., C₁-C₆ for straight chain, C₃-C₆ for branched chain), for example four or fewer. Likewise, certain cycloalkyls have from three to eight carbon atoms in their ring structure, such as five or six carbons in the ring structure.

The term “substituted alkyls” refers to alkyl moieties having substituents replacing a hydrogen on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “aralkyl” moiety is an alkyl substituted with an aryl (e.g., phenylmethyl(benzyl)). If not otherwise indicated, the terms “alkyl” and “lower alkyl” include linear, branched, cyclic, unsubstituted, substituted, and/or heteroatom-containing alkyl or lower alkyl, respectively.

The term “alkenyl” refers to a linear, branched or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, cyclopentenyl, cyclohexenyl, cyclooctenyl, and the like. Generally, although again not necessarily, alkenyl groups can contain 2 to about 18 carbon atoms, and more particularly 2 to 12 carbon atoms. The term “lower alkenyl” refers to an alkenyl group of 2 to 6 carbon atoms, and the specific term “cycloalkenyl” intends a cyclic alkenyl group, preferably having 5 to 8 carbon atoms. The term “substituted alkenyl” refers to alkenyl substituted with one or more substituent groups, and the terms “heteroatom-containing alkenyl” and “heteroalkenyl” refer to alkenyl or heterocycloalkenyl (e.g., heterocylcohexenyl) in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl, respectively.

The term “alkynyl” refers to a linear or branched hydrocarbon group of 2 to 24 carbon atoms containing at least one triple bond, such as ethynyl, n-propynyl, and the like. Generally, although again not necessarily, alkynyl groups can contain 2 to about 18 carbon atoms, and more particularly can contain 2 to 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The terms “alkyl”, “alkenyl”, and “alkynyl” are intended to include moieties which are diradicals, i.e., having two points of attachment. A nonlimiting example of such an alkyl moiety that is a diradical is —CH₂CH₂—, i.e., a C₂ alkyl group that is covalently bonded via each terminal carbon atom to the remainder of the molecule.

The term “alkoxy” refers to an alkyl group bound through a single, terminal ether linkage; that is, an “alkoxy” group may be represented as —O-alkyl where alkyl is as defined above. A “lower alkoxy” group intends an alkoxy group containing 1 to 6 carbon atoms, and includes, for example, methoxy, ethoxy, n-propoxy, isopropoxy, t-butyloxy, etc. Preferred substituents identified as “C₁-C₆ alkoxy” or “lower alkoxy” herein contain 1 to 3 carbon atoms, and particularly preferred such substituents contain 1 or 2 carbon atoms (i.e., methoxy and ethoxy).

The term “aryl” refers to an aromatic substituent containing a single aromatic ring or multiple aromatic rings that are fused together, directly linked, or indirectly linked (such that the different aromatic rings are bound to a common group such as a methylene or ethylene moiety). Aryl groups can contain 5 to 20 carbon atoms, and particularly preferred aryl groups can contain 5 to 14 carbon atoms. Examples of aryl groups include benzene, phenyl, pyrrole, furan, thiophene, thiazole, isothiazole, imidazole, triazole, tetrazole, pyrazole, oxazole, isooxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, napthridine, indole, benzofuran, purine, benzofuran, deazapurine, or indolizine. Those aryl groups having heteroatoms in the ring structure may also be referred to as “aryl heterocycles”, “heterocycles,” “heteroaryls” or “heteroaromatics”. The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino (including alkylamino, dialkylamino, arylamino, diaryl amino, and alkylaryl amino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings, which are not aromatic so as to form a multicyclic system (e.g., tetralin, methylenedioxyphenyl). If not otherwise indicated, the term “aryl” includes unsubstituted, substituted, and/or heteroatom-containing aromatic substituents.

The terms “heterocyclyl” or “heterocyclic group” include closed ring structures, e.g., 3- to 10-, or 4- to 7-membered rings, which include one or more heteroatoms. “Heteroatom” includes atoms of any element other than carbon or hydrogen. Examples of heteroatoms include nitrogen, oxygen, sulfur and phosphorus.

Heterocyclyl groups can be saturated or unsaturated and include pyrrolidine, oxolane, thiolane, piperidine, piperazine, morpholine, lactones, lactams, such as azetidinones and pyrrolidinones, sultams, and sultones. Heterocyclic groups such as pyrrole and furan can have aromatic character. They include fused ring structures, such as quinoline and isoquinoline. Other examples of heterocyclic groups include pyridine and purine. The heterocyclic ring can be substituted at one or more positions with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, or an aromatic or heteroaromatic moiety. Heterocyclic groups can also be substituted at one or more constituent atoms with, for example, a lower alkyl, a lower alkenyl, a lower alkoxy, a lower alkylthio, a lower alkylamino, a lower alkylcarboxyl, a nitro, a hydroxyl, —CF₃, or —CN, or the like.

The term “halo” or “halogen” refers to fluoro, chloro, bromo, and iodo. “Counterion” is used to represent a small, negatively charged species such as fluoride, chloride, bromide, iodide, hydroxide, acetate, and sulfate. The term sulfoxide refers to a sulfur attached to 2 different carbon atoms and one oxygen and the S—O bond can be graphically represented with a double bond (S═O), a single bond without charges (S—O) or a single bond with charges [S(+)—O(−)].

The terms “substituted” as in “substituted alkyl,” “substituted aryl,” and the like, as alluded to in some of the aforementioned definitions, is meant that in the alkyl, aryl, or other moiety, at least one hydrogen atom bound to a carbon (or other) atom is replaced with one or more non-hydrogen substituents. Examples of such substituents include, without limitation: functional groups such as halo, hydroxyl, silyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), C₂-C₂₄ alkylcarbonato (—O—(CO)—O-alkyl), C₆-C₂₀ arylcarbonato (—O—(CO)—O-aryl), carboxy (—COOH), carboxylato (—COO—), carbamoyl (—(CO)—NH₂), mono-(C₁-C₂₄ alkyl)-substituted carbamoyl (—(CO)—NH(C₁-C₂₄ alkyl)), di-(C₁-C₄ alkyl)-substituted carbamoyl (—(CO)—N(C₁-C₂₄ alkyl)₂), mono-substituted arylcarbamoyl (—(CO)—NH-aryl), thiocarbamoyl (—(CS)—NH₂), carbamido (—NH—(CO)—NH₂), cyano (—CN), isocyano (—N⁺C⁻), cyanato (—O—CN), isocyanato (—ON⁺C⁻), isothiocyanato (—S—CN), azido (—N═N⁺═N⁻), formyl (—(CO)—H), thioformyl (—(CS)—H), amino (—NH₂), mono- and di-(C₁-C₂₄ alkyl)-substituted amino, mono- and di-(C₅-C₂₀ aryl)-substituted amino, C₂-C₂₄ alkylamido (—NH—(CO)-alkyl), C₆-C₂₀ arylamido (—NH—(CO)-aryl), imino (—CR═NH where R═hydrogen, C₁-C₂₄ alkyl, C₅-C₂₀ aryl, C₆-C₂₄ alkaryl, C₆-C₂₄ aralkyl, etc.), alkylimino (—CR═N (alkyl), where R═hydrogen, alkyl, aryl, alkaryl, etc.), arylimino (—CR═N (aryl), where R═hydrogen, alkyl, aryl, alkaryl, etc.), nitro (—NO₂), nitroso (—NO), sulfo (—SO₂—OH), sulfonato (—SO₂—O—), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), phosphono (—P(O)(OH)₂), phosphonato (—P(O)(O—)₂), phosphinato (—P(O)(O—)), phospho (—PO₂), and phosphino (—PH₂); and the hydrocarbyl moieties C₁-C₂₄ alkyl, C₂-C₂₄ alkenyl, C₂-C₂₄ alkynyl, C₅-C₂₀ aryl, C₆-C₂₄ alkaryl, and C₆-C₂₄ aralkyl.

In addition, the aforementioned functional groups may, if a particular group permits, be further substituted with one or more additional functional groups or with one or more hydrocarbyl moieties such as those specifically enumerated above. Analogously, the above-mentioned hydrocarbyl moieties may be further substituted with one or more functional groups or additional hydrocarbyl moieties such as those specifically enumerated.

When the term “substituted” appears prior to a list of possible substituted groups, it is intended that the term apply to every member of that group. For example, the phrase “substituted alkyl, alkenyl, and aryl” is to be interpreted as “substituted alkyl, substituted alkenyl, and substituted aryl.” Analogously, when the term “heteroatom-containing” appears prior to a list of possible heteroatom-containing groups, it is intended that the term apply to every member of that group. For example, the phrase “heteroatom-containing alkyl, alkenyl, and aryl” is to be interpreted as “heteroatom-containing alkyl, substituted alkenyl, and substituted aryl.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

The terms “stable compound” and “stable structure” are meant to indicate a compound that is sufficiently robust to survive isolation, and as appropriate, purification from a reaction mixture, and formulation into an efficacious therapeutic agent.

The terms “free compound” is used herein to describe a compound in the unbound state.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

The term “small molecule” is an art-recognized term. In certain embodiments, this term refers to a molecule, which has a molecular weight of less than about 2000 amu, or less than about 1000 amu, and even less than about 500 amu.

All percentages and ratios used herein, unless otherwise indicated, are by weight.

Embodiments described herein relate to compositions and methods of attenuating and/or treating opioid withdrawal and/or dependence in a subject in need thereof, and particularly relates to the use of thiol-based compounds in compositions and methods of attenuating and/or treating opioid withdrawal and/or dependence.

Opioids are substances that act by binding to opioid receptors, which receptors are found principally in the central and peripheral nervous system and the gastrointestinal tract. These receptors mediate both the psychoactive and the somatic effects of opioids. Medically opioids are primarily used for pain relief, including anesthesia. Other medical uses include suppression of diarrhea and suppressing cough.

Opioids include opiates, which are alkaloid compounds naturally found in the opium poppy plant (i.e., Papaver somniferum). The psychoactive compounds found in the opium plant include opium, heroin, morphine, codeine and thebaine. Examples of synthetic, or semi-synthetic, opioids include hydrocodone; oxycodone; fentanyl; methadone; pethidine and hydromorphone. Opioid therapy is the treatment of a human subject with opioids, typically a prolonged treatment with opioids, typically to achieve analgesic effects.

In some embodiments, the methods described herein include the administration of a pharmaceutical composition to a human subject, in need thereof, in an amount which is effective to inhibit the physical dependence on opioids by the subject. A human subject in need thereof is a human who is to receive, or is receiving, opioid therapy. Administration includes administration by a physician or by self-administration.

Physical dependence on an opioid is a state of adaptation by a patient who has received the opioid for a period of time and who experiences, or would experience, withdrawal syndrome if the opioid is abruptly withdrawn or if a narcotic antagonist (e.g., naloxone) is administered. Physical dependence is a normal physiological response.

Opioid withdrawal or opioid withdrawal syndrome includes symptoms which may range from mild to severe, depending on how dependent the subject is on the opioid. Dependency can be directly tied to the length of time taking an opioid, dosage amount, which particular opioid was taken, route of administration, underlying medical conditions, mental health, and certain biological and environmental factors, such as family history of addiction, previous trauma, and stressful surroundings.

Withdrawal from an opioid may roughly adhere to the following timeline, although it can vary from subject to subject. Early withdrawal symptoms typically start within 6-12 hours after last dose is taken for short-acting opioids, and start within 30 hours after last dose is taken for longer-acting opioids. Early withdrawal symptoms include: lacrimation, muscle aches, agitation, insomnia, excessive yawning, anxiety, panic, rhinorrhea, sweating, tachycardia, hypertension and fever. Late withdrawal symptoms typically peak within 72 hours after last dose is taken, usually lasting about a week, and include nausea and vomiting, diarrhea, piloerection, stomach cramps, depression, and drug cravings. Other withdrawal symptoms may include, for example, mydriasis, restlessness; tremor; involuntary movements; muscle twitches; abdominal cramps; cold flashes; substantial physical and mental fatigue; dysphoric mood; drowsiness; salivation; loss of appetite; headache; dizziness; fainting; malaise; shivering; muscle/joint pain; irritability; poor concentration; confusion; flu-like symptoms; and the like.

In some embodiments, administration of the thiol based compounds described herein to a subject undergoing and/or about to undergo opioid withdrawal and/or exhibiting or experiencing opioid withdrawal symptoms, such as subject dependent or addicted to an opioid, can prevent, attenuate, and/or treat opioid withdrawal symptoms.

In some embodiments, the thiol based compound that is used to prevent, attenuate, and/or treat opioid withdrawal symptoms can include a D-cysteine ester, a cystine ester, or an adduct, a pharmaceutically acceptable salt, a tautomer, or a solvate thereof.

In some embodiments, the D-cysteine ester can be a D-cysteine alkyl ester that includes a compound having the structure of formula:

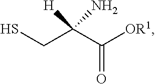

or a pharmaceutically acceptable salt, tautomer, or solvate thereof, where R¹ is a lower alkyl (e.g., C₁-C₆ alkyl).

In other embodiments, R¹ is selected from the group consisting of methyl, ethyl, propyl, and butyl. In certain embodiments, the cysteine alkyl ester can be a D-cysteine ethyl ester, prodrug thereof, or pharmaceutically acceptable salt, tautomer, or solvate thereof.

In some embodiments, the pharmaceutical salt of a D-cysteine alkyl ester can include a hydrochloride salt.

In still other embodiment, the adduct of the D-cysteine alkyl ester can be a biologically active adduct and include at least one of an albumin adduct, a glucose adduct, an L-cysteine adduct, an L-glutathione adduct, or an S-nitroso adduct

In other embodiments, the cystine ester can have the formula:

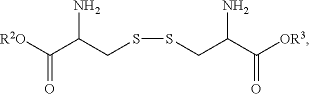

or an adduct, a pharmaceutically acceptable salt, a tautomer, or a solvate thereof; where R² and R³ are the same or different and are selected from the group consisting of H, unsubstituted or substituted C₁-C₂₄ alkyl, C₂-C₂₄ alkenyl, C₂-C₂₄ alkynyl, C₃-C₂₀ aryl, heterocycloalkenyl containing from 5-6 ring atoms (wherein from 1-3 of the ring atoms is independently selected from N, NH, N(C₁-C₆ alkyl), NC(O)(C₁-C₆ alkyl), O, and S), heteroaryl containing from 5-14 ring atoms, (wherein from 1-6 of the ring atoms is independently selected from N, NH, N(C₁-C₃ alkyl), O, and S), and heterocyclyl containing from 5-14 ring atoms (wherein from 1-6 of the ring atoms is independently selected from N, NH, N(C₁-C₃ alkyl), O, and S), and at least one of R² and R³ is not a H; or an adduct, a pharmaceutically acceptable salt, a tautomer, or a solvate thereof.

In some embodiments, R² and R³ are independently H or an unsubstituted or substituted C₁-C₂₄ alkyl, wherein at least one of R² and R³ is not a H. In other embodiments, R² and R³ are independently selected from the group consisting of H, methyl, ethyl, propyl, and butyl, and at least one of R² and R³ is not a H.

In other embodiments, the cystine ester can be a cystine dialkyl ester, or an adduct, a pharmaceutically acceptable salt, a tautomer, or a solvate thereof. The cystine dialkyl ester can comprise a mixture at least one of D or L isomers of a cystine dialkyl ester. For example, the cystine dialkyl ester can comprise a mixture of: less than about 50% by weight of the D isomer of a cystine dialkyl ester and greater than about 50% by weight of the L isomer of a cystine dialkyl ester, less than about 25% by weight of the D isomer of a cystine dialkyl ester and greater than about 75% by weight of the L isomer of a cystine dialkyl ester, less than about 10% by weight of the D isomer of a cystine dialkyl ester and greater than about 90% by weight of the L isomer of a cystine dialkyl ester, less than about 1% by weight of the D isomer of a cystine dialkyl ester and greater than about 99% by weight of the L isomer of a cystine dialkyl ester, greater than about 50% by weight of the D isomer of a cystine dialkyl ester and less than about 50% by weight of the L isomer of a cystine dialkyl ester, greater than about 75% by weight of the D isomer of a cystine dialkyl ester and less than about 25% by weight of the L isomer of a cystine dialkyl ester, greater than about 90% by weight of the D isomer of a cystine dialkyl ester and less than about 10% by weight of the L isomer of a cystine dialkyl ester, or greater than about 99% by weight of the D isomer of a cystine dialkyl ester and less than about 1% by weight of the L isomer of a cystine dialkyl ester.

In a still further embodiment, the cystine dialkyl ester can consist essentially of or consist of the D isomer of cystine dialkyl ester. In yet another embodiment, the cystine dialkyl ester can consist essentially of or consist of the L isomer of cystine dialkyl ester.

In some embodiments, the cystine dialkyl ester is a D-cystine dialkyl ester, or an adduct, a pharmaceutically acceptable salt, a tautomer, or a solvate thereof. Advantageously, it was found that D-isomer can be more active than the corresponding L-isomer of the cystine dialkyl ester and unlike L-cysteine does not increase upper airway resistance or promote cystinosis-like effects in animals or have negative cardiovascular effects of L-cysteine esters. The D-cystine dialkyl ester can be selected from the group consisting of D-cystine dimethyl ester, D-cystine diethyl ester, combinations thereof, or an adduct, a pharmaceutically acceptable salt, a tautomer, or a solvate thereof.

In some embodiments, the adduct of the cystine ester can be a biologically active adduct and include at least one of an albumin adduct, a glucose adduct, an L-cysteine adduct, an L-glutathione adduct, or an S-nitroso adduct.

Compositions comprising a D-cysteine ester, a cystine ester, or an adduct, a pharmaceutically acceptable salt, a tautomer, or a solvate thereof described herein can be administered to a subject prior to or during opioid induced dependence and/or withdrawal. In some embodiments, the composition is administered to a human subject, in need thereof, during opioid therapy, optionally, slightly before the commencement of opioid therapy. For example, administration is begun at most about 48 hours before the first dose of an opioid or at the time of the first dose of an opioid, and is substantially continued for the duration of the opioid therapy. Alternatively, administration can be begun at any point during opioid therapy.

In some embodiments, administration of a composition comprising a D-cysteine ester, a cystine ester, or an adduct, a pharmaceutically acceptable salt, a tautomer, or a solvate thereof can inhibit, reduce, prevent, and/or shorten the duration of opioid dependence in the subject. That is, the methods described herein are considered to be effective if they cause one or more of: a reduction/prevention of dependence on an opioid and/or shortening of the duration of any dependence to an opioid. For example, dependence would be inhibited if upon cessation of opioid therapy, withdrawal symptoms are inhibited.

Inhibition of dependence can be assessed by comparing the magnitude and/or duration of dependence in a subject at two different occasions, that is, i) when administered the pharmaceutical composition during an opioid therapy; and ii) when not administered the pharmaceutical composition during an opioid therapy. An assessment is made as to the severity of withdrawal symptoms once the opioid is discontinued at the different occasions.

Inhibition of dependence can also be assessed by comparing the magnitude and/or duration of dependence in different subjects being treated with the same opioid, some of whom were administered the pharmaceutical composition during a therapy and some whom were not administered the pharmaceutical composition during a therapy. An assessment is made as to the severity of withdrawal symptoms once the opioid is discontinued between the different subjects.

Typically, dependence and/or withdrawal symptoms can be inhibited by at least about 10%, at least about 20%, at least about 30%, at least about 40%, at least about 50%, at least about 60%, at least about 70%, at least about 80%, at least about 90%, or about 100%.

In some embodiments, the subject treated with a composition comprising a D-cysteine ester, a cystine ester, or an adduct, a pharmaceutically acceptable salt, a tautomer, or a solvate thereof is a neonatal subject has or at risk of neonatal opioid withdrawal syndrome (NOWS). (NOWS) is a subset of neonatal abstinence syndrome (NAS) and refers to neonatal withdrawal from opioid drugs and can occur in the presence of other drug withdrawal syndromes. NOWS is a generalized multisystem disorder that predominantly involves the central and autonomic nervous systems and the gastrointestinal tract. In the United States, neonatal withdrawal following in-utero opioid exposure is typically the result of prolonged maternal use and/or abuse of illicit or prescribed opioids during pregnancy. Birth leads to the abrupt cessation of fetal substance exposure and can precipitate acute withdrawal symptoms that can result in severe health complications with prolonged recovery and longer hospitalization. Withdrawal can be both severe and intense, with an estimated 60% to 80% of exposed neonates requiring pharmacological intervention to control symptoms (see Kraft, Walter K.; Stover, Megan W.; Davis, Jonathan M. Neonatal abstinence syndrome: Pharmacologic strategies for the mother and infant Seminars in Perinatology 40.3 (Apr. 1, 2016): 203-212, and Tolia V N, Patrick S W, Bennett M M, et al. Increasing incidence of the neonatal abstinence syndrome in U.S. neonatal ICUs. N Engl J Med. 2015; 372 (22): 2118-2126, which are incorporated herein by reference in their entireties).

While the signs and symptoms of NOWS are similar to those experienced by adults undergoing acute opiate withdrawal, they present a higher risk to the neonate due to the infant's dependence on others for all aspects of well-being.

NOWS presenting signs include central nervous system hyperirritability (tremors, jitteriness, irritability, hyperactive muscle reflexes, and excessive high-pitched cry), autonomic nervous system deregulation and instability (tachypnea, nasal flaring, hyperphagia, temperature instability, insomnia, sweating, mottle skin, yawning, and sneezing), and gastrointestinal symptoms (diarrhea, vomiting, and poor feeding) (see Hudak M L, Tan R C. The Committee on Drugs and the Committee on Fetus and Newborn. Neonatal Drug Withdrawal. Pediatrics. 2012; 129; e540, and Kocheriakota P. Neonatal Abstinence Syndrome. Pediatrics. 2014; 134 (2): e547-561, which are incorporated herein by reference in their entireties). Seizures are generally rare, although they have been reported occurring in 2% to 11% of neonate cases in the early stage of severe opioid withdrawal (see Doberczak T M, Kandall S R, Wilets I. Neonatal opiate abstinence syndrome in term and preterm infants. J Pediatr 1991; 118:933-7, which is incorporated herein by reference in its entirety, especially if medical treatment has been delayed. A recent large observational cohort study of Medicaid babies in 46 US States reported an incidence of seizures was 2.7% among the 1705 observed cases of NAS (see Desai, R. J., Hernandez-Diaz, S., Bateman, B. T. & Huybrechts, K. F. Increase in prescription opioid use during pregnancy among Medicaid-enrolled women. Obstet. Gynecol. 123, 997-1002 (2014), which is incorporated herein by reference in its entirety. Neonates with NOWS can also experience weight loss or failure to thrive, which often results from a combination of poor feeding, vomiting, nausea, and diarrhea (see Kocheriakota P. Neonatal Abstinence Syndrome. Pediatrics. 2014; 134 (2): e547-561, which is incorporated herein by reference in its entirety). If left untreated, some cases of NOWS may even lead to death. Some of the less severe signs and symptoms of opiate withdrawal may persist for several months (see Hudak M L, Tan R C. The Committee on Drugs and the Committee on Fetus and Newborn. Neonatal Drug Withdrawal. Pediatrics. 2012; 129; e540, which is incorporated herein by reference in its entirety)

In some embodiments, administration of a composition including the D-cysteine ester, the cystine ester, or an adduct, a pharmaceutically acceptable salt, a tautomer, or a solvate thereof can inhibit, reduce, prevent, and/or shorten the duration of NOWS in a neonatal subject.

In some embodiments, compositions including the D-cysteine ester, cystine ester, or adduct, a pharmaceutically acceptable salt, tautomer, or solvate thereof can be administered to the subject in combination with at least one additional compound, agent, and/or therapeutic agent useful for treating the subject or opioid use, opioid dependence, and/or opioid withdrawal. These additional compounds, agents, and/or therapeutic agents can include commercially available agents or compounds, known to treat, prevent, or reduce opioid use, opioid dependence, and/or opioid withdrawal in the subject.

In some embodiments, the at least one additional therapeutic agent can include an opioid antagonist, such as naloxone (naloxone, chemically known as 1-N-allyl-14-hydroxynordihydromorphinone). Naloxone can block the euphorigenic activity of an opioid and eliminate the development of psychological dependence. The inhibition of opiate effects by naloxone also prevents the development of physical dependence. U.S. Pat. No. 3,773,955 incorporated herein by reference, describes the oral combination of naloxone with a number of opiates particularly methadone. U.S. Pat. No. 4,457,933 describes the protection with naloxone of oral dosage forms of various opioids against both oral and parenteral abuse. U.S. Pat. No. 4,661,492 mentions the incorporation of 1-3 mg of naloxone in an oral unit dose of buprenorphine (2 mg).

In some embodiments, the compositions include an oxymorphol analog of naloxone, a naloxone salt, or a naloxone dihydrate.

Other compositions can include naltrexone (1-N-cyclopropylmethyl-14-hydroxynordihydromorphinone). Naltrexone is a pure opiate antagonist which, when administered orally as a maintenance drug for opiate addicts, blocks the effects of self-administered opiates thereby contributing to the extinction of drug craving.

In some embodiments, the composition can include nalmefene (also known as nalmetrene), another opioid antagonist. Nalmefene is similar in both structure and activity to naltrexone. Reported advantages of nalmefene relative to naltrexone include longer half-life, greater oral bioavailability and no observed dose-dependent liver toxicity. As with other opioid antagonists of the sort, nalmefene may precipitate acute withdrawal symptoms in patients who are dependent on opioid drugs, or post-operatively, to counteract the effects of strong opioids used in surgery. Any other opioid antagonist that can potentiate or enhance the effectiveness of a composition including the D-cysteine ester, cystine ester, or adduct, pharmaceutically acceptable salt, tautomer, or solvate thereof is contemplated.

In still other embodiments a composition including the D-cysteine ester, cystine ester, or adduct, pharmaceutically acceptable salt, tautomer, or solvate thereof can include an additional agent selected from an opioid, doxapram and enantiomers thereof, acetazolamide, almitrine, theophylline, caffeine, methylprogesterone and related compounds, sedatives that decrease arousal threshold in sleep disordered breathing patients, sodium oxybate, benzodiazepine receptor agonists, orexin antagonists, tricyclic antidepressants, serotonergic modulators, adenosine and adenosine receptor and nucleoside transporter modulators, cannabinoids, orexins, melatonin agonists, ampakines, or combinations thereof.

In one embodiment, the composition including the D-cysteine ester, cystine ester, or adduct, pharmaceutically acceptable salt, tautomer, or solvate thereof described herein and an additional agent are physically mixed in the composition. In another embodiment, the composition a composition including the D-cysteine ester, a cystine ester, or an adduct, a pharmaceutically acceptable salt, a tautomer, or a solvate thereof described herein and the additional agent are physically separated in the composition.

In some embodiments, a composition including the D-cysteine ester, cystine ester, or adduct, pharmaceutically acceptable salt, tautomer, or solvate thereof described herein may be packaged with at least one opioid capable of inducing dependence or addiction in a subject. The amount of the D-cysteine ester, cystine ester, or adduct, pharmaceutically acceptable salt, tautomer, or solvate thereof described herein can be effective to prevent the opioid induced dependence or withdrawal.

In some embodiments, an effective amount (i.e., dose) of the D-cysteine ester, cystine ester, or adduct, pharmaceutically acceptable salt, tautomer, or solvate thereof described herein to be administered to a subject can be determined depending upon, for example, age, body weight, symptom, the desired therapeutic effect, the route of administration, and the duration of the treatment. Exemplary doses can be from about 0.01 to about 1000 mg, by oral administration. Examples of dose ranges can include from a minimum dose of about 0.01, 0.10, 0.50, 1, 5, 10, 25, 50, 100, 125, 150, 200, or 250 mg to a maximum dose of about 300, 400, 500, 600, 700, 800, 900, or 1000 mg, wherein the dose range can include from any one of the foregoing minimum doses to any one of the foregoing maximum doses. Specific examples of particular effective amounts contemplated via oral administration can include about 0.02, 0.03, 0.04, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000 mg or more. The oral dose can be administered once daily, twice daily, three times daily, or more frequently.

The dose of the cysteine ester, cystine ester, or adduct, pharmaceutically acceptable salt, tautomer, or solvate thereof for use in parenteral administration (e.g., intravenous administration) is generally from about 0.01 to about 300 mg/kg body weight. Examples of dose ranges can include from a minimum dose of about 0.01, 0.10, 0.50, 1, 5, 10, 25, 50, or 100 mg/kg body weight to a maximum dose of about 125, 150, 175, 200, 250, 275, or 300 mg/kg body weight, wherein the dose range can include from any one of the foregoing minimum doses to any one of the foregoing maximum doses. Specific examples of effective amounts contemplated include about 0.02, 0.03, 0.04, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300 mg/kg body weight or more. Continuous intravenous administration is also contemplated for from 1 to 24 hours per day to achieve a target concentration from about 0.01 mg/L blood to about 100 mg/L blood. Exemplary dose ranges can include from a minimum dose of about 0.01, 0.10, 0.25, 0.50, 1, 5, 10, or 25 mg/L blood to a maximum dose of about 40, 45, 50, 55, 60, 65, 70, 75, 80, 85, 90, or 100 mg/L, wherein an exemplary dose ranges can include from any one of the foregoing minimum doses to any one of the foregoing maximum doses. Specific examples of particular effective amounts contemplated via this route include about 0.02, 0.03, 0.04, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 mg/L blood or more. The dose to be used can depend upon various conditions, and there may be cases wherein doses lower than or greater than the ranges specified above are used.

In some embodiments, the composition is administered concurrently with opioid administration and/or up to about 10 minutes, up to about 20 minutes, up to about 30 minutes, up to about 40 minutes, up to about 50 minutes, up to about 60 minutes, up to about 70 minutes, up to about 80 minutes, up to about 90 minutes, up to about 100 minutes, up to about 110 minutes, or up to about 120 minutes before or after initiation of opioid administration.

The D-cysteine ester, cystine ester, or adduct, pharmaceutically acceptable salt, tautomer, or solvate thereof described herein may be administered in the form of, for example, solid compositions, liquid compositions, or other compositions for oral administration, injections, liniments, or suppositories for parenteral administration. Solid compositions for oral administration include compressed tablets, pills, capsules, dispersible powders, and granules. Capsules include hard capsules and soft capsules. In such solid compositions, the cysteine ester, cystine ester, or adduct, pharmaceutically acceptable salt, tautomer, or solvate thereof can be admixed with an excipient (e.g., lactose, mannitol, glucose, microcrystalline cellulose, or starch), combining agents (e.g., hydroxypropyl cellulose, polyvinyl pyrrolidone, or magnesium metasilicate aluminate), disintegrating agents (e.g., cellulose calcium glycolate), lubricating agents (e.g., magnesium stearate), stabilizing agents, agents to assist dissolution (e.g., glutamic acid or aspartic acid), or the like. The agents may, if desired, be coated with coating agents (e.g., sugar, gelatin, hydroxypropyl cellulose, or hydroxypropylmethyl cellulose phthalate), or be coated with two or more films. Further, coating may include containment within capsules of absorbable materials such as gelatin.

Liquid compositions for oral administration include pharmaceutically acceptable solutions, suspensions, emulsions, syrups, and elixirs. In such compositions, the cysteine ester, cystine ester, or adduct, pharmaceutically acceptable salt, tautomer, or solvate thereof is dissolved, suspended, or emulsified in a commonly used diluent (e.g., purified water, ethanol, or mixture thereof). Furthermore, such liquid compositions may also comprise wetting agents, suspending agents, emulsifying agents, flavoring agents (e.g., flavor-masking agents) sweetening agents, perfuming agents, preserving agents, buffer agents, or the like.

Injections for parenteral administration include solutions, suspensions, emulsions, and solids, which are dissolved or suspended. For injections, the cysteine ester, cystine ester, or adduct, pharmaceutically acceptable salt, tautomer, or solvate thereof can be dissolved, suspended, and/or emulsified in a solvent. The solvents are, for example, distilled water for injection, physiological salt solution, vegetable oil, propylene glycol, polyethylene glycol, alcohol such as ethanol, or a mixture thereof. Moreover the injections also can include stabilizing agents, agents to assist dissolution (e.g., glutamic acid, aspartic acid, or POLYSORBATE 80), suspending agents, emulsifying agents, soothing agents, buffer agents, preserving agents, etc. The compositions are sterilized in the final process or manufactured and prepared by sterile procedure. The compositions also can be manufactured in the form of sterile solid compositions, such as a freeze-dried composition, and can be sterilized or dissolved immediately before use in sterile distilled water for injection or some other solvent.

Other compositions for parenteral administration include liquids and ointments for external use, endermic liniments, compositions for inhalation, sprays, suppositories for rectal administration, and pessaries for vaginal administration, which compositions include a cystine ester and are administered by methods known in the art.

Compositions comprising the cysteine ester, cystine ester, or adduct, pharmaceutically acceptable salt, tautomer, or solvate thereof for inhalation or sprays may comprise additional substances other than diluents, such as, e.g., stabilizing agents (e.g., sodium sulfite hydride), isotonic buffers (e.g., sodium chloride, sodium citrate or citric acid). See, for example, the methods described in U.S. Pat. Nos. 2,868,691 and 3,095,355. The cysteine ester, cystine ester, or adduct, pharmaceutically acceptable salt, tautomer, or solvate thereof can be effectively distributed by inhalation or spray using a self-propelling composition that includes a solution or dispersion of the cysteine ester, cystine ester, or adduct, pharmaceutically acceptable salt, tautomer, or solvate thereof in micronized form. For example, an effective dispersion of finely divided drug particles can be accomplished with the use of very small quantities of a suspending agent, present as a coating on micronized drug particles. Evaporation of the propellant from the aerosol particles after spraying from the aerosol container leaves finely divided drug particles coated with a fine film of the suspending agent. In the micronized form, the average particle size can be less than about 5 microns. The propellant composition can employ, as the suspending agent, a fatty alcohol such as oleyl alcohol. Propellants that may be employed include hydrofluoroalkane propellants and chlorofluorocarbon propellants. Dry powder inhalation also can be employed.

The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology and pharmaceutics. In general, such preparatory methods include the step of bringing the active ingredient into association with a carrier or one or more other accessory ingredients, and then, if necessary or desirable, shaping or packaging the product into a desired single-dose or multi-dose unit.

Although the descriptions of pharmaceutical compositions provided herein are principally directed to pharmaceutical compositions, which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions is contemplated include, but are not limited to, humans and other primates, mammals including commercially relevant mammals such as cattle, pigs, horses, sheep, cats, and dogs.

The regimen of administration may affect what constitutes an effective amount. The therapeutic formulations may be administered to the patient either prior to or after the onset of acute or post-acute withdrawal. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds employed in the pharmaceutical composition at levels lower than that required in order to achieve the desired therapeutic effect and gradually increase the dosage until the desired effect is achieved.

The invention is further illustrated by the following example, which is not intended to limit the scope of the claims.

### Example 1

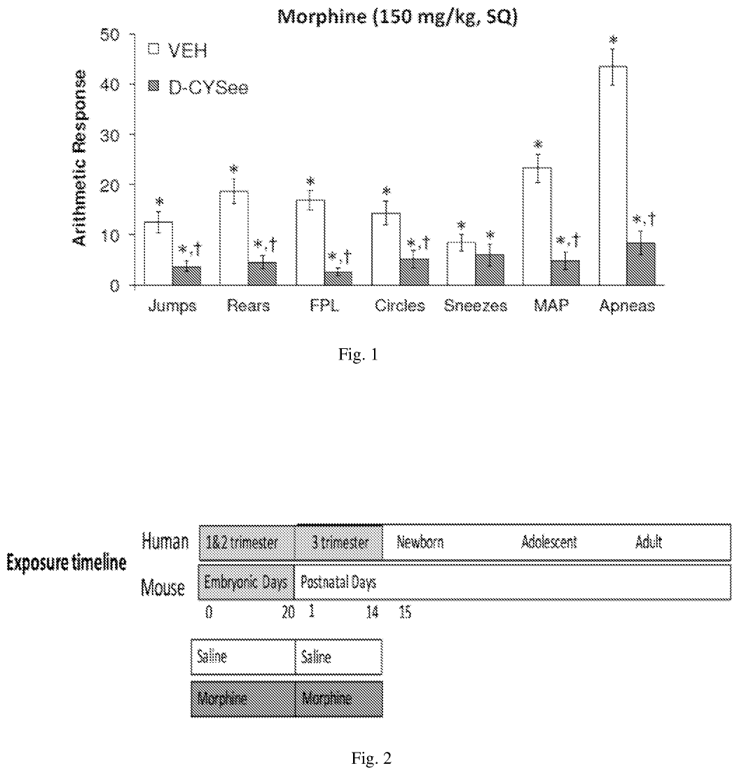

FIG. 1 illustrates that D-CYSee markedly diminishes withdrawal responses in morphine dependent rats and may modulate addiction. Rats received a depot subcutaneous injection of morphine (150 mg/kg) with or without continuous infusion of D-CYSee (20 μmol/kg/hour, IV) and one day later the rats received an intraperitoneal injection of naloxone HCl (1.5 mg/kg) to elicit withdrawal responses. The data in FIG. 1 shows that D-CYSee attenuated opioid withdrawal symptoms including jumping behavior, episodes of fore-paw licking, episodes of sneezing, change in mean arterial blood pressure, rearing behavior, circling behavior, and episodes of apneas.

### Example 2

FIG. 2 illustrates a schematic timeline of an assay of neonatal morphine exposure of female mice for determining neonatal opioid withdrawal syndrome (NOWS). Female mice are implanted with osmotic mini-pumps containing 20 mg/kg/day morphine sulfate and mated. Offspring are injected twice daily with 10 mg/kg morphine for 14 days.

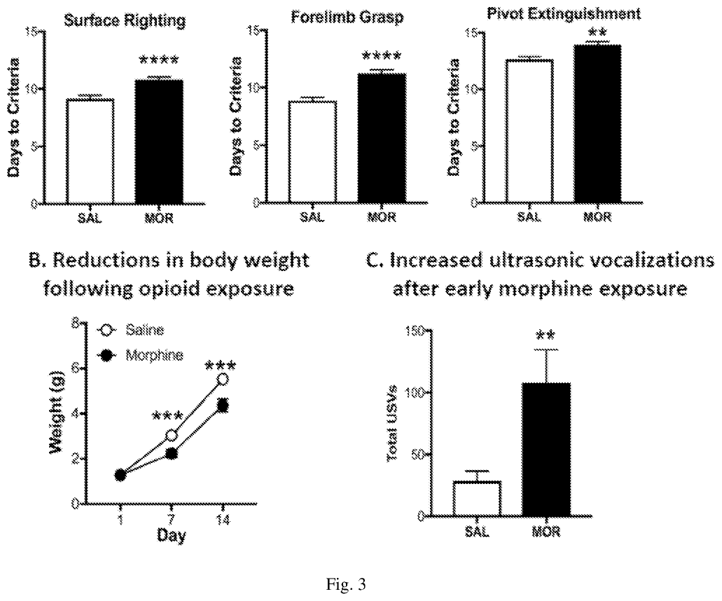

FIG. 3 illustrates graphs and a plot showing developmental milestones are impaired following early opioid exposure. The morphine-exposed mice exhibit (A) delays in reaching specific developmental milestones independent of sex, (B) reduced body weight gain, and (C) increased vocalization compared to saline exposed pups 24 h after cessation of drug treatment. Data are mean±SEM. * P<0.05, ** P<0.01, *** P<0.001 compared to saline-treated animals.

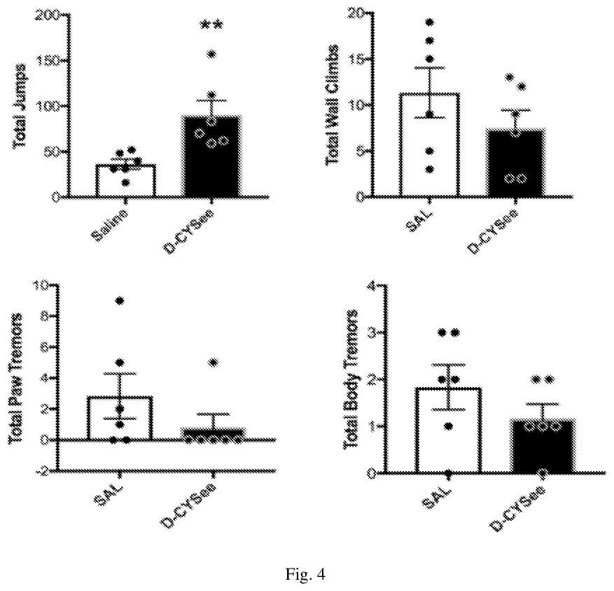

FIG. 4 illustrates a graph showing D-CYSee modulates NOWS. Mouse pups exhibit increases in somatic signs of opioid withdrawal including jumps (top left panel), wall climbs (top right), paw tremors (bottom left) and body tremors (bottom right). D-CYSee (30 mg/kg s.c.) given prior to naloxone (1.0 mg/kg) reduces all somatic signs of withdrawal except for jumping behavior, which is enhanced.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1-29. (canceled)

30. A method of treating opioid withdrawal and/or dependence in a subject in need thereof, the method comprising:
administering to the subject a therapeutically effective amount of a composition comprising a D-cysteine ester, a cystine ester, or an adduct, a pharmaceutically acceptable salt, a tautomer, or a solvate thereof.

31. The method of claim 30, wherein the subject has an opioid dependence or addiction.

32. The method of claim 30, wherein the subject has neonatal opioid withdrawal syndrome.

33. The method of claim 30, wherein the D-cysteine ester, adduct, pharmaceutically acceptable salt, tautomer, or solvate thereof includes a compound having the structure of formula: 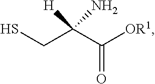
or a pharmaceutically acceptable salt, tautomer, or solvate thereof; where R¹ is an unsubstituted or substituted lower alkyl.

34. The method of claim 30, wherein the D-cysteine ester, adduct, pharmaceutically acceptable salt, tautomer, or solvate thereof comprises a D-cysteine ethyl ester, an adduct thereof, or a pharmaceutically acceptable salt, tautomer, or solvate thereof.

35. The method of claim 30, wherein the adduct of the D-cysteine ester comprises at least one of an albumin adduct, a glucose adduct, an L-cysteine adduct, an L-glutathione adduct, or an S-nitroso adduct.

36. The method of claim 30, wherein the cystine ester, adduct, pharmaceutically acceptable salt, tautomer, or solvate thereof includes a compound having the structure of formula: 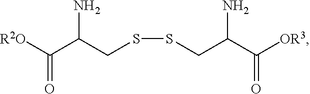
or an adduct, a pharmaceutically acceptable salt, a tautomer, or a solvate thereof; where R² and R³ are the same or different and are each selected from H, an unsubstituted or substituted C₁-C₂₄ alkyl, C₂-C₂₄ alkenyl, C₂-C₂₄ alkynyl, C₃-C₂₀ aryl, heterocycloalkenyl containing from 5-6 ring atoms, heteroaryl, and heterocyclyl containing from 5-14 ring atoms, wherein at least one of R² and R³ is not a H.

37. The method of claim 36, wherein R² and R³ are each independently H or an unsubstituted or substituted C₁-C₂₄ alkyl, wherein at least one of R² and R³ is not a H.

38. The method of claim 36, wherein R² and R³ are each independently selected from H, methyl, ethyl, propyl, or butyl, wherein at least one of R² and R³ is not a H.

39. The method of claim 30, wherein the cystine ester, adduct, pharmaceutically acceptable salt, tautomer, or solvate thereof is a cystine dialkyl ester, adduct, pharmaceutically acceptable salt, tautomer, or solvate thereof.

40. The method of claim 39, wherein the cystine dialkyl ester is a D-cystine dialkyl ester, adduct, pharmaceutically acceptable salt, tautomer, or solvate thereof.

41. The method of claim 39, wherein the cystine dialkyl ester, adduct, pharmaceutically acceptable salt, tautomer, or solvate thereof is selected from cystine dimethyl ester, cystine diethyl ester, combinations thereof, or an adduct, a pharmaceutically acceptable salt, a tautomer, or a solvate thereof.

42. The method of claim 39, wherein the cystine dialkyl ester, adduct, pharmaceutically acceptable salt, tautomer, or solvate thereof is a D-cystine dimethyl ester or an adduct, a pharmaceutically acceptable salt, a tautomer, or a solvate thereof.

43. The method of claim 30, wherein the opioid comprises morphine and/or fentanyl.

44. The method of claim 30, further comprising administering an opioid antagonist to the subject.

45. The method of claim 44, wherein the opioid antagonist is naloxone, an oxymorphol analog of naloxone, a naloxone salt, or a naloxone dihydrate.

46. The method of claim 44, wherein the opioid antagonist is naloxone.

47. The method of claim 45, wherein the oxymorphol analog of naloxone is naltrexone.

48. A method of treating neonatal opioid withdrawal syndrome in a subject in need thereof, the method comprising:
administering to the subject a therapeutically effective amount of a composition comprising a D-cysteine ester, a cystine ester, or an adduct, a pharmaceutically acceptable salt, a tautomer, or a solvate thereof.
